(19) United States
(12) Patent Application Publication
(10) Pub. No.: US 2003/0073918 A1
(43) Pub. Date: Apr. 17, 2003

(54) Configuration-retaining conduit for use in obtaining metabolic measurements from respiration

(76) Inventors: Joseph A. Orr, Park City, UT (US); Scott A. Kofoed, Bountiful, UT (US); Kevin Durst, Salt Lake City, UT (US)

Correspondence Address: TRASK BRITT, P.O. BOX 2550, SALT LAKE CITY, UT, 84110, US

(21) Appl. No.: 09/977,754

(22) Filed: Oct. 15, 2001

(51) Int. Cl. A61B 5/103; A61B 5/117; A61B 5/08

(52) U.S. Cl. 600/531

(57) ABSTRACT

A conduit for use in establishing flow communication between a breathing apparatus, such as a mouthpiece of mask, and a metabolic analyzer, such as that used for determining the resting metabolic rate (RMR) of an individual. At least a section if the conduit may be bent or otherwise manipulated into a desired configuration and substantially retain the desired configuration until being remanipulated. Such a desired configuration may at least partially support the breathing apparatus while an individual is in a resting position. Manipulable regions of the conduit may be formed by longitudinally collapsible and expandable tubing, such as corrugated tubing, from tubing that carries elongate compliant members that may be bent and will retain a desired configuration, from viscoelastic tubing, or otherwise, as known in the art. Methods for establishing communication between an individual's airway and a metabolic analyzer are also disclosed, as are methods for effecting RMR analyses.

### BACKGROUND OF THE INVENTION

1. Field of the Invention

The present invention relates generally to apparatus that are used in obtaining metabolic measurements by way of an individual's respiration and, more specifically, to breathing apparatus, such as a mouthpiece or mask, through which an individual may breathe and conduits coupled thereto to facilitate monitoring of the individual's respiration. In particular, the present invention relates to conduits that may be formed by an individual into a desired shape and that will retain such a shape and may support a breathing apparatus in position relative to a patient at rest. The present invention also relates to breathing apparatus for use in obtaining metabolic measurements from an individual's respiration.

2. Background of Related Art

Indirect calorimetry is a method of assessing metabolic rate by measuring the amount of oxygen consumed. Since every metabolic activity requires oxygen, there is a direct relationship between calories spent and oxygen consumed. Metabolic rate measurements are traditionally done at exercise to determine the amount of calories spent at various levels of exertion, as well as during rest to determine an individual's resting metabolic rate (RMR). RMR, which is an indicator of how many calories would be consumed if an individual were to spend an entire day resting, is measured in calories per day. Although it is an unlikely scenario that an individual would spend an entire day resting, about 70-80% of each day is spent at a metabolic rate near the resting level. Resting metabolic rate is useful in determining the ideal caloric consumption rate for the individual to achieve weight loss.

To obtain an individual's true RMR measurement by use of indirect calorimetry, it is essential to analyze all of the individual's expired air. When a mouthpiece is used to collect the expired air, respiration through the individual's nose is inhibited. Thus, to prevent suffocation, it is also desirable for the individual to inhale through the mouthpiece. Accordingly, mouthpieces that are used in obtaining RMR measurements may be fitted with at least a pair of one-way valves. These one-way valves control the flow of air into and out of the mouthpiece, at least one valve permitting fresh air to enter the mouthpiece, while at least one other valve of the mouthpiece is configured to cause exhaled air to flow into the conduit. The mouthpiece, in turn, conveys air that has been expired by the individual through a conduit, typically referred to as a “breathing hose”, and into an instrument that measures gas flow and oxygen concentration. Based on the measured gas flow and oxygen concentration values, the individual's oxygen consumption and metabolic rate may be determined.

A valid RMR measurement requires between about 3 and about 30 minutes for the patient to arrive at a truly resting state and for the measured oxygen levels and gas flows to stabilize. Since the resting metabolic rate should be measured at rest, the individual should not be required to hold or support the mouthpiece or conduit.

The conduits that have typically been used to facilitate metabolic analysis by way of patient's respiration are somewhat undesirable from the standpoint that they do not provide any support to breathing apparatus, such as mouthpieces or masks, that are coupled thereto. In addition, some slack may remain in conventional conduits when coupled between a breathing apparatus that has been secured to the patient's face and a metabolic analyzer. Consequently, these conventional conduits may become entangled with wires or tubing and may also get in the way of healthcare professionals that are working with or around the patient.

It would, therefore, be desirable to provide a conduit for use with a breathing apparatus and which is capable of substantially retaining a desired shape. Such a conduit with a length that may be readily tailored would also be desirable. It would also be desirable to provide a self-supporting conduit that can be adjusted to at least partially hold a breathing apparatus in place, without requiring a patient to actively hold the conduit or breathing apparatus in a desired position during the RMR test. The inventors are not, however, aware of the use of any such conduits with breathing apparatus.

### SUMMARY OF THE INVENTION

The present invention includes a conduit that is configured to couple a mouthpiece, mask, or other breathing apparatus configured to communicate with the airway of a patient to an instrument that measures gas flow and oxygen concentration, as known in the art. A conduit incorporating teachings of the present invention may be bent into a desired configuration and substantially retain that configuration. As such, the conduit may at least partially hold the mouthpiece, mask, or other breathing apparatus into position while the patient is at rest.

An exemplary embodiment of the conduit comprises a length of longitudinally collapsible, or “corrugated” tubing, which has an accordion-like appearance and, accordingly, may be at least partially collapsed or expanded along the length thereof. In addition, such a length of longitudinally collapsible and expandable tubing may be bent, with an outer bent edge being somewhat longitudinally expanded and an inner bent edge being somewhat longitudinally collapsed. Preferably, the material from which the longitudinally collapsible and expandable length of the conduit is formed, as well as the configuration of the longitudinally collapsible and expandable length of the conduit, permit that portion of the conduit to retain the desired shape into which it is placed. When a conduit incorporating teachings of the present invention includes a longitudinally collapsible and expandable length of tubing, the longitudinally collapsible and expandable length of tubing may form substantially the entire length of the conduit or only a portion of the conduit's length.

Alternatively, a conduit according to the present invention may comprise a length of tubing having a substantially uniform cross-section taken transversely to the length thereof and which is capable of being bent into a desired configuration and substantially retaining that configuration. By way of example, tubing with one or more wires, metal strips, or other elongate compliant members extending lengthwise along the wall of the conduit (internally, on a surface of the wall, or some combination thereof) may be used to facilitate bending of the tube into a desired configuration and substantially retention, or maintenance, of such configuration during use. As another example, tubing that may be bent into a desired configuration and, upon being heated, retain that configuration may be used as all or part of a conduit incorporating teachings of the present invention. In still another example, a so-called viscoelastic material, which is flexible, but will substantially retain a desired shape, may be used to form all or part of the length of a conduit according to the present invention.

The conduit may be removably coupled or irremovably secured to a breathing apparatus, such as a mouthpiece or breathing mask. A breathing apparatus incorporating teachings of the present invention may include a first, inlet valve that facilitates the flow of “fresh” air therein and, thus, the inspiration of such air by a patient during inhalation. Such a breathing apparatus also includes a second, outlet valve which permits expired air to enter a conduit that has been coupled to the breathing apparatus. The outlet valve and a conduit-coupling element of the breathing apparatus may be located at a bottom portion of the breathing apparatus so as to cause the conduit to extend over a patient's chest during RMR evaluation. The patient's chest may, therefore, support the conduit, which may be bent into a position so as to prevent movement thereof from the patient's chest, as well as to extend upwardly somewhat from the patient's chest towards the patient's mouth. The conduit may, in turn, support the breathing apparatus, retaining the same in position during use.

A conduit incorporating teachings of the present invention, as well as the mouthpiece or other breathing apparatus used therewith, are preferably disposable and, thus, formed from relatively inexpensive materials and by relatively inexpensive processes. Alternatively, the conduit and breathing apparatus may be formed from materials that will withstand desired sterilization processes, thus facilitating their reuse.

Other features and advantages of the present invention will become apparent to those of ordinary skill in the art through consideration of the ensuing description, the accompanying drawings, and the appended claims.

### BRIEF DESCRIPTION OF THE DRAWINGS

In the drawings, which depict exemplary embodiments of various aspects of the present invention:

FIG. 1 schematically represents an exemplary embodiment of a mouthpiece according to the present invention and an exemplary embodiment a conduit coupled thereto;

FIG. 2 is a cross-section taken along line 2-2 of FIG. 1;

FIG. 3 is a cross-sectional representation that depicts the conduit of FIG. 1 in a longitudinally extended orientation;

FIG. 4 is a cross-sectional representation that depicts the conduit of FIG. 1 in a longitudinally collapsed orientation;

FIG. 5 is a side view showing the conduit of FIG. 1 in a bent orientation;

FIG. 6 depicts the conduit of FIG. 1 in a second exemplary supporting orientation upon an individual's abdomen;

FIG. 7 is a schematic representation of another exemplary embodiment of respiratory conduit incorporating teachings of the present invention that may be formed into and substantially maintained in a desired configuration;

FIG. 8 schematically depicts yet another exemplary embodiment of respiratory conduit of the present invention;

FIG. 9 schematically depicts still another exemplary embodiment of respiratory conduit of the present invention, in which at least a central section of the conduit may be formed into and substantially maintained in a desired configuration; and

FIG. 10 schematically depicts another embodiment of breathing apparatus to which a respiratory conduit according to the present invention may be coupled.

### DETAILED DESCRIPTION

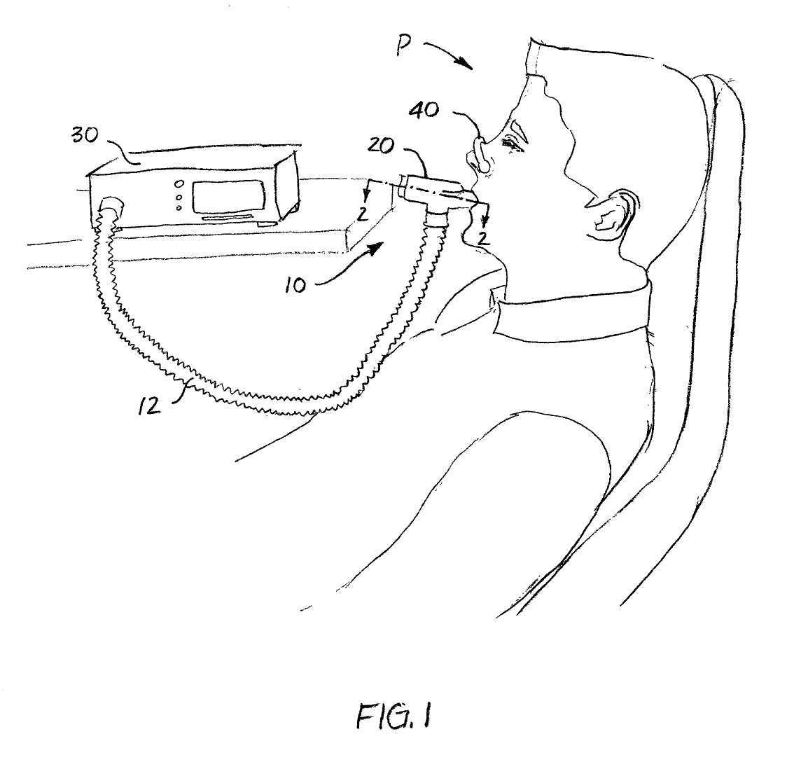

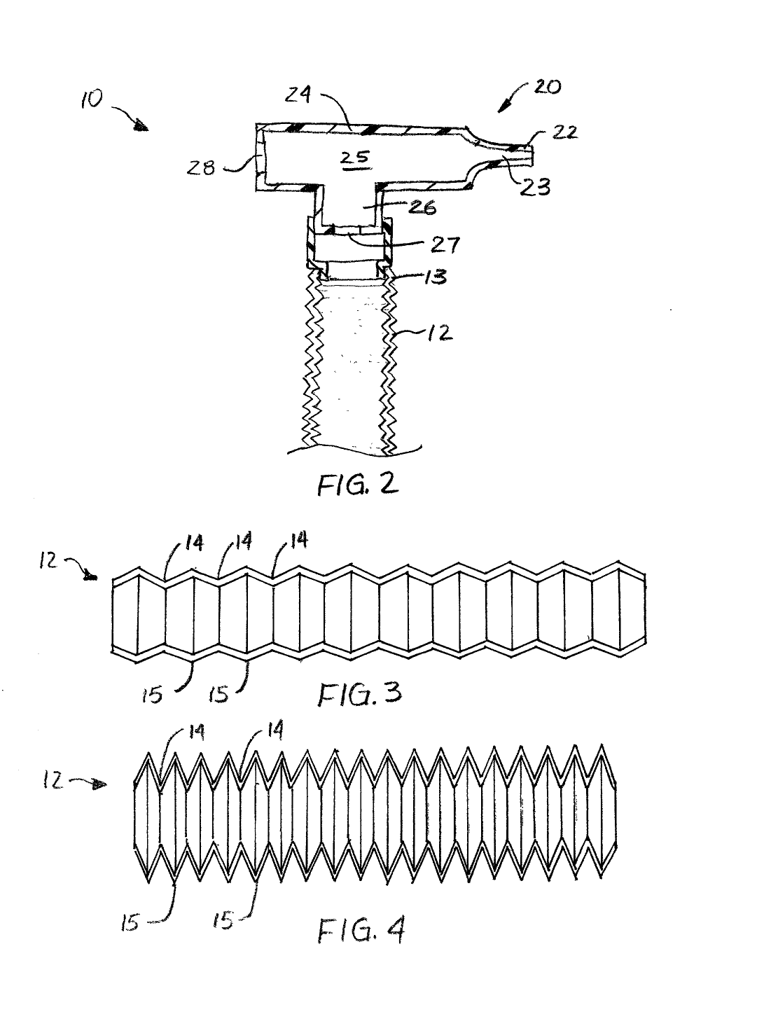

With reference to FIGS. 1 and 2, a patient P is permitted to assume a resting position (e.g., lying down or sitting in a reclined position) to facilitate evaluation of the patient's RMR. A breath collection system 10 is then placed in communication with the patient's airway and coupled to a metabolic analyzer 30 of a known type, such as an apparatus for measuring respiratory flow and oxygen content. As depicted, breath collection system 10 includes a conduit 12 that is configured to be coupled to metabolic measurement instrument 30 and a breathing apparatus, depicted as a mouthpiece 20, which is coupled to conduit 12 and is configured to communicate with an airway (not shown) of patient P.

Mouthpiece 20 includes a breathing end 22, a body 24, and a conduit coupling section 26 that communicates with body 24. Each of these elements include hollow interiors so as to facilitate the flow of respiratory gases therethrough.

Breathing end 22 of mouthpiece 20, which includes a breathing aperture 23 formed therethrough, is configured to be comfortably received at least partially within the mouth of patient P. In the depicted example of mouthpiece 20, breathing end 22 thereof comprises an extension of body 24, having a substantially common axis therewith. Breathing aperture 23 communicates with a hollow interior 25 of body 24 and, thus, establishes communication between the airway (not shown) of patient P and the interior of mouthpiece 20.

A first, inlet valve 28 is positioned on mouthpiece 20 (e.g., on body 24 thereof) to permit communication between the hollow interior thereof and the environment external to mouthpiece 20 while patient P inhales. By way of example only, inlet valve 28 may comprise a one-way valve of a type known in the art that permits “fresh” air to enter hollow interior 25 of body 24 upon the formation of a negative pressure therein, such as when patient P inhales.

Conduit coupling section 26 is configured to receive or to be received by an end portion of a conduit 12 according to the present invention. Conduit coupling section 26 may be located along the bottom of body 24 and extend in a direction transverse to the primary axis of body 24, as depicted. Alternatively, conduit coupling section 26 may be located elsewhere on body 24 of mouthpiece 20, such as at an end of body 24. As another alternative, mouthpiece 20 may include more than one conduit coupling section 26, such as one at the bottom of body 24 and another at an end thereof.

Communication between the interior of mouthpiece 20 and conduit 10 may be selectively established by way of a second, outlet valve 27. As an example, outlet valve 27 may comprise a one-way valve of a known type that permits exhaled air to pass therethrough only upon the formation of a positive pressure within hollow interior 25 of body 24, such as when patient P exhales. Outlet valve 27 may be positioned on conduit coupling section 26 or on conduit 12, at a coupling end 13 thereof. In embodiments of mouthpiece 20 that include more than one conduit coupling section 26, inlet valve 28 and outlet valve 27 may both be positioned at a corresponding conduit coupling section 26. In addition, inlet valve 28 and outlet valve 27 of such a mouthpiece may both be removable from body 24 (e.g., by sliding or screwing into a corresponding valve-retaining element of body 24) and interchangeable with one another to facilitate such alternate positioning of a conduit 12 on mouthpiece 20.

Coupling end 13 of conduit 12 may be secured to conduit coupling section 26 of mouthpiece 20 by any suitable means, such as by interference fit between complementarily configured male and female ends of these members, by way of a hose clamp that surrounds coupling end 13 of conduit 12 to be positioned on and, thus, receive, conduit coupling section 26 of mouthpiece 20, or by complementary threading on an exterior or interior of coupling end 13 of conduit 12 and an interior or exterior of conduit coupling section 26. Alternatively, coupling end 13 of conduit 12 may be pre-secured to conduit coupling section 26 of mouthpiece 20, such as by use of a suitable glue, cement, or other adhesive. Preferably, coupling end 13 of conduit 12 couples to conduit coupling section 26 in a substantially fluid-tight fashion. Accordingly, the air exhaled by patient P may be conveyed to metabolic analyzer 30 without being diluted by the air external to conduit 12 and mouthpiece 20. In addition, the pressure with which air is exhaled by patient P may be substantially maintained within mouthpiece 20 and throughout the length of conduit 12.

The coupling of conduit 12 to a corresponding feature of metabolic analyzer 30 may be effected by similar means, and is also preferably substantially fluid-tight.

With continued reference to FIGS. 1 and 2, a first exemplary embodiment of conduit 12 incorporating teachings of the present invention comprises a so-called “corrugated” section of tubing. Thus, conduit 12 includes a series of alternating constricted regions 14 and enlarged regions 15. Corrugated tubing is available from a variety of vendors, including Qosina, of 150-Q Executive Drive, Edgewood, N.Y.( as part number T6009) and Cleveland Tubing, of Cleveland, Tenn. (as part number 23072).

With reference to FIG. 3, conduit 12 is shown in a longitudinally expanded, or extended, orientation, wherein constricted regions 14 thereof have been moved away from and, thus, are positioned between adjacent enlarged regions 15.

As depicted in FIGS. 4 and 5, conduit 12 may be fully or partially longitudinally collapsed by compressing one or more constricted regions 14 or portions thereof in close proximity to one or both of the adjacent enlarged regions 15. Accordingly, conduit 12 may be at least partially collapsed along the length thereof, or substantially longitudinally, as shown in FIG. 4, or partially longitudinally collapsed and/or expanded into a bent configuration, as depicted in FIG. 5.

Conduit 12 may be substantially longitudinally collapsed, as shown in FIG. 4, to minimize the amount of space needed for packaging, shipping, and storage. Sections of conduit 12 may likewise be substantially longitudinally collapsed to provide conduit 12 with a desired length, such as to remove slack therefrom during use in establishing fluid communication between a mouthpiece 20 or other breathing apparatus and a metabolic analyzer 30, as depicted in FIG. 1.

Conduit 12 maybe partially longitudinally collapsed and/or expanded or formed into a bent configuration, as depicted in FIG. 5, during use thereof with mouthpiece 20 or another breathing apparatus. When conduit 12 is bent, it may substantially retain its bent shape until re-bent to form a new shape. It is preferred that conduit 12 have sufficient strength and rigidity to at least partially support mouthpiece 20 or another breathing apparatus while substantially retaining a desired shape, such as that shown in FIG. 1.

Referring again to FIG. 1, in a first exemplary supporting orientation, conduit 12 extends downward relative to mouthpiece 12 and is bent such that a section thereof rests upon the chest of patient P. Another section of conduit 12 is bent in such a manner as to extend from the chest of patient P to metabolic analyzer 30. While patient P's chest supports conduit 12, the rigidity of conduit 12, as well as its ability to substantially maintain the desired, bent configuration, may partially support mouthpiece 20 in position relative to the mouth of patient P. In the first exemplary supporting orientation, one or more of the sections of conduit 12 may also be longitudinally collapsed and/or expanded to tailor the length thereof between mouthpiece 20 and metabolic analyzer 30.

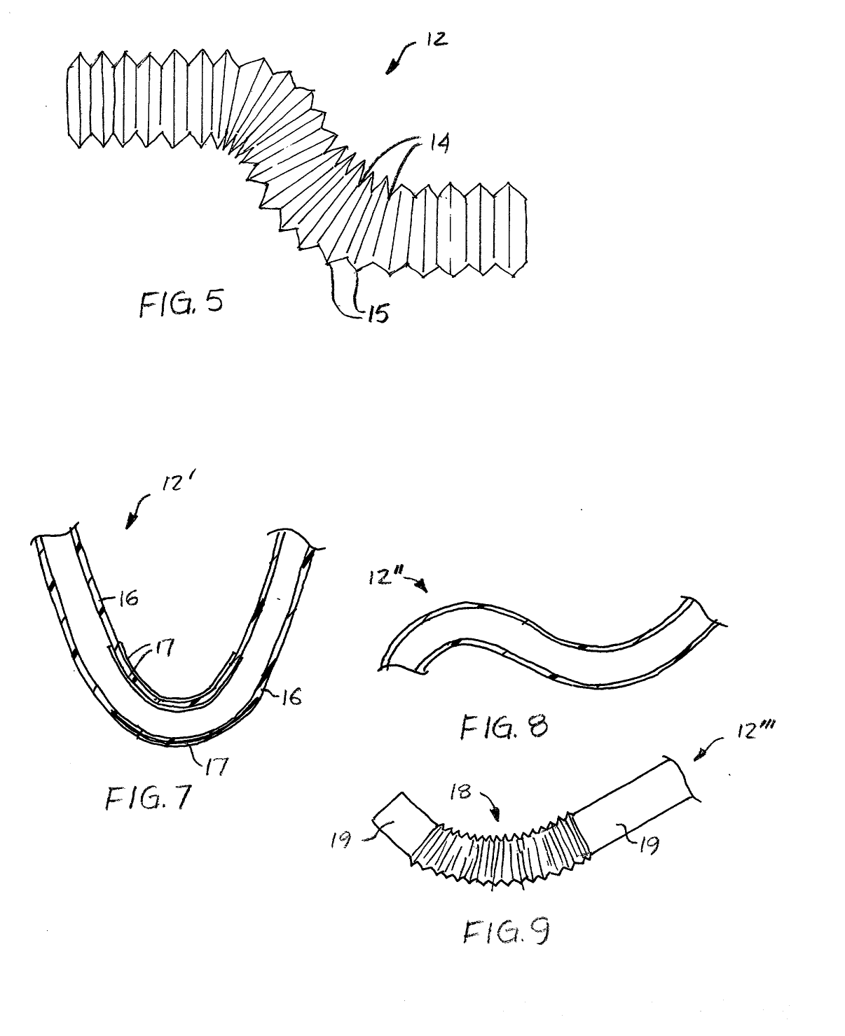

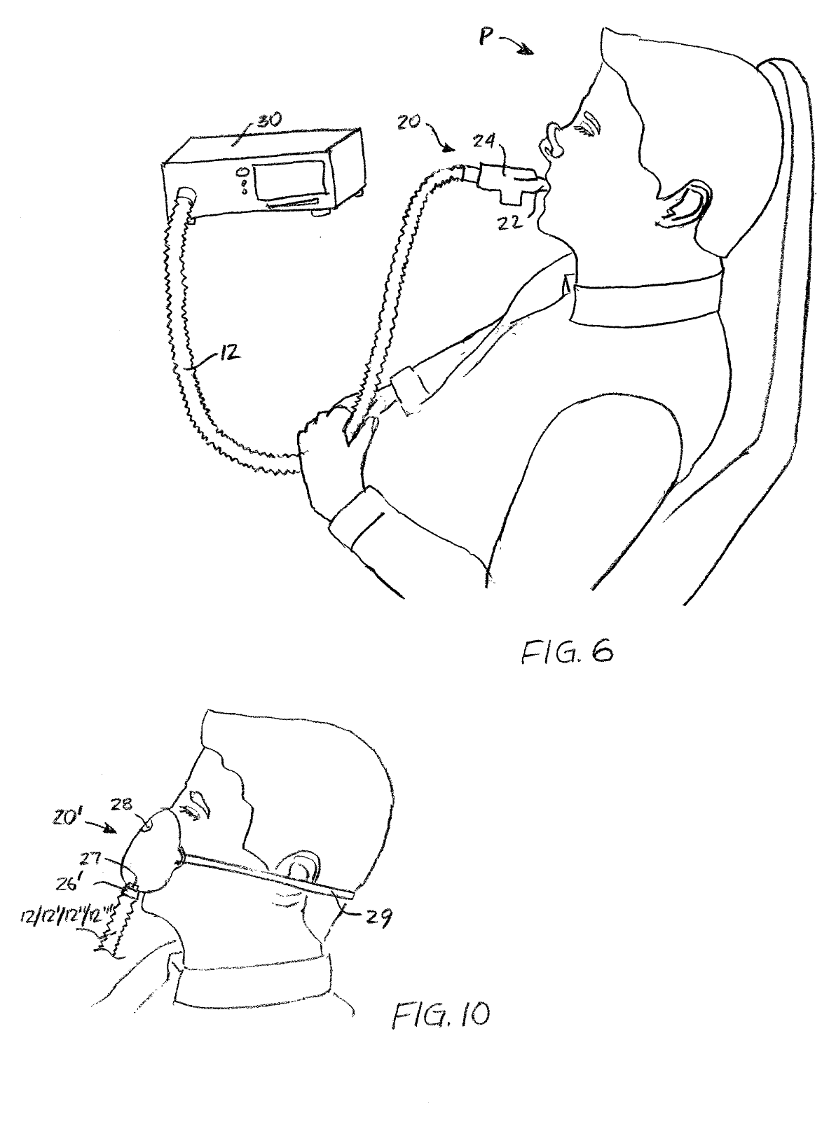

FIG. 6 depicts conduit 12 bent into a second exemplary supporting orientation upon the chest of a patient P that may at least partially support a breathing apparatus, again a mouthpiece 20, that has been placed in communication with an airway (not shown) of patient P. In FIG. 6, conduit 12 is depicted as being secured to a conduit coupling section 26 located at an end of body 24 opposite from that at which breathing end 22 is located. Accordingly, a first section of conduit 12 extends outward somewhat, then is bent and extends downward, toward the abdomen of patient P. Another section of conduit 12 rests upon patient P's abdomen, while yet another section of conduit 12 extends between the abdomen of patient P and a metabolic analyzer 30. As shown, the supporting orientation of conduit 12 that is depicted in FIG. 6 permits patient P to hold conduit 12 at a location at or near where conduit 12 contacts his or her abdomen, while allowing the hands of patient P to remain in a resting position upon his or her abdomen.

In FIG. 7, another exemplary embodiment of conduit 12′ incorporating teachings of the present invention is illustrated. The cross section of conduit 12′ taken transverse to the length thereof is substantially constant along the length of conduit 12′. Conduit 12′ includes one or more elongate compliant members 17 carried by a wall 16 thereof and extending at least partially along the length thereof. Compliant members 17, which may comprise lengths of wire, strips of metal or another compliant material, or the like, may be carried upon an exterior or interior surface of wall 16 or internally within wall 16. Each compliant member 17 may be bent into a desired configuration, thus causing conduit 12′ to be bent, and may substantially maintain the desired configuration, causing conduit 12′ to substantially maintain a desired configuration therefor.

Yet another embodiment of conduit 12″ according to the present invention is depicted in FIG. 8. Conduit 12″ is formed from a material that substantially retains a shape into which it is formed, which materials are commonly referred to as “viscoelastic” materials, and has a substantially uniform cross section taken transverse to the length thereof. Alternatively, conduit 12″ may be formed from a material that will substantially retain a desired configuration upon placement of conduit 12″ in the desired configuration and application of heat to at least bent locations of conduit 12″.

As shown in FIG. 9, another embodiment of conduit 12′″ that is within the scope of the present invention includes a configurable section 18 that may be formed into and will maintain a desired configuration, with at least one other section 19 thereof comprising tubing that has conventionally been used in analyzing the RMR of a patient. As depicted, configurable section 18 of conduit 12′″ comprises a longitudinally collapsible and expandable, corrugated section of tubing, having the characteristics described herein in reference to FIGS. 3-5, although configurable sections of other manipulable, shape-retaining configurations may also be used. The remainder of conduit (i.e., section 19), comprises a conventional, flexible, noncollapsible tube that is not configured to maintain a desired, bent or linear configuration.

FIG. 10 depicts the use of a different type of breathing apparatus, a mask 20′, with a conduit 12, 12′, 12″, 12′″ secured thereto to convey air that has been exhaled by a patient to a metabolic analyzer 30. When mask 20′ is used, it is not necessary to inhibit the patient's respiration through his or her nose, as respiration from both the nose and mouth of the patient may communicate with an interior 25′ of mask 20′.

Mask 20′ includes at least one conduit coupling section 26′ that facilitates securing of conduit 12, 12′, 12 ″, 12′″ thereto, such as by an interference fit, complementary threading, the use of a hose clamp, or otherwise, as known in the art. Alternatively, conduit 12, 12′, 12″, 12′″ may be secured to conduit coupling section 26′ of mask 20′ by way of a suitable adhesive. An outlet valve 27 communicates with conduit coupling section 26′, while an inlet valve 28 is positioned elsewhere on mask 20′ so as to permit “fresh” air to enter mask 20′ as a patient inhales.

Mask 20′ may also include a position retention member 29, such as a strip of elastic, to at least partially maintain the position of mask 20′ upon the face of a patient, as well as to substantially prevent air from entering or exiting mask 20 at locations between the mask and the patient's face. Of course, a conduit incorporating teachings of the present invention (e.g., conduit 12, 12′, 12″, or 12′″) may also partially support mask 20′ in position upon the user's face.

Turning again to FIG. 1, an example of a method for obtaining metabolic measurements from a patient P, such as patient P's RMR, includes having patient P lie down or sit in a reclined fashion.

Conduit 12 may be coupled at one end 13 to mouthpiece 20, mask 20′ (FIG. 10), or another breathing apparatus and at an opposite end to a corresponding feature on a metabolic analyzer 30 of a known type. Such coupling may be effected by any means known in the art, such as the interference fit, threading, or clamping discussed previously herein. Again, it is preferred that fluid-tight connections be made between conduit 12 and both the breathing apparatus (i.e., mouthpiece 20, mask 20′ (FIG. 10), etc.) and metabolic analyzer 30.

The length of conduit 12 (or of conduit 12′″ or any other expandable conduit incorporating teachings of the present invention) may be tailored by fully or partially expanding conduit 12 from a longitudinally collapsed configuration, or by longitudinally collapsing portions of conduit 12. The configuration of conduit 12 (or of any of conduits 12′, 12″, 12′″, or any other conduit incorporating teachings of the present invention) may be configured by selectively bending sections thereof, preferably while conduit 12 rests upon the chest or abdomen of patient P or another supporting surface (e.g., a table, bed, chair, etc.). In the case of conduits 12 and 12′″, such selective bending includes causing sections of conduits 12, 12′″ to be at least partially longitudinally expanded on an outer bend edge 0 and more longitudinally collapsed or compressed along an inner bend edge I. The configuration into which conduit 12 is placed, as well as the rigidity of conduit 12 and its ability to substantially retain a desired shape, will preferably provide at least partial support to a mouthpiece 20, a mask 20′ (FIG. 10), or another breathing apparatus that is placed in flow communication with the mouth of patient P.

Communication between the airway (not shown) of a patient P and a metabolic analyzer 30 may be established by placing a breathing apparatus, such as mouthpiece 20 or mask 20′ (FIG. 10), in flow communication with the mouth of patient P. Preferably, a fluid-tight seal is formed between the breathing apparatus and patient P's mouth so as to prevent a drop in pressure within the breathing apparatus and dilution of the respiratory gases exhaled by patient P by “fresh” air external to the breathing apparatus. Mouthpiece 20, mask 20′ (FIG. 10) or another breathing apparatus may be placed in flow communication with the mouth of patient P either before or after conduit 12 is coupled to metabolic analyzer 30 or formed or placed into a desired configuration.

If the nose of patient P is not in flow communication with the breathing apparatus, as is the case when mouthpiece 20 or another mouthpiece is used, airflow through patient P's nose may be restricted, as known in the art, such as by use of a nose clip 40, as depicted.

Once a breathing apparatus, such as mouthpiece 20 or mask 20′ (FIG. 10), has been positioned and properly supported in such a manner (e.g., at least partially by conduit 12, 12′, 12″, 12′″) as to allow patient P to rest, metabolic analysis of patient P's exhalate, such as is required in determining the RMR of patient P, may be conducted, as known in the art.

Although the foregoing description contains many specifics, these should not be construed as limiting the scope of the present invention, but merely as providing illustrations of some exemplary embodiments. Similarly, other embodiments of the invention may be devised which do not depart from the spirit or scope of the present invention. Features from different embodiments may be employed in combination. The scope of the invention is, therefore, indicated and limited only by the appended claims and their legal equivalents, rather than by the foregoing description. All additions, deletions, and modifications to the invention, as disclosed herein, which fall within the meaning and scope of the claims are to be embraced thereby.

What is claimed:

1. A breath collection system for use in obtaining metabolic measurements from an individual's respiration, comprising:
a breathing apparatus configured to communicate with at least a mouth of the individual; and
a conduit including a first end coupled to said mouthpiece and a second end configured to be coupled to apparatus for monitoring the individual's respiration, said conduit including at least a section that is configured to be placed into a desired configuration and that substantially maintains said desired configuration until placed in another desired configuration.

2. The system of claim 1, wherein said breathing apparatus comprises a mouthpiece.

3. The system of claim 2, wherein said mouthpiece comprises a breathing end configured to be at least partially inserted into the mouth of the individual.

4. The system of claim 3, wherein said mouthpiece comprises a conduit coupling section oriented in an at least partially downwardly extending direction relative to said breathing end, said conduit-receiving extension being configured to be coupled to said first end of said conduit.

5. The system of claim 1, wherein said breathing apparatus comprises a mask configured to be placed over at least the mouth of the individual.

6. The system of claim 1, wherein said breathing apparatus comprises: at least one inlet valve; and at least one outlet valve.

7. The system of claim 6, wherein said at least one inlet valve comprises a one-way valve that facilitates introduction of gases to be inhaled by the individual into said breathing apparatus.

8. The system of claim 6, wherein said at least one inlet valve opens upon application of a negative pressure within said breathing apparatus.

9. The system of claim 6, wherein said at least one outlet valve comprises a one-way valve that facilitates evacuation of the individual's expiratory gases from said breathing apparatus.

10. The system of claim 9, wherein said at least one outlet valve opens upon application of a positive pressure within said breathing apparatus.

11. The system of claim 10, wherein said at least one outlet valve is positioned on said at least one of a conduit coupling section of said breathing apparatus and an end of said conduit.

12. The system of claim 1, wherein at least said section of said conduit comprises a longitudinally expandable and collapsible member.

13. The system of claim 12, wherein said longitudinally expandable and collapsible member comprises a section of corrugated tubing.

14. The system of claim 1, wherein at least said section of said conduit carries at least one elongate compliant member.

15. A breathing conduit, comprising:
a first end configured to be coupled to a breathing apparatus that is capable of communicating with at least a mouth of an individual;
a second end configured to be coupled to apparatus for monitoring the individual's respiration; and
at least a section located between said first end and said second end and which is configured to be placed into a desired configuration and that substantially maintains said desired configuration until placed in another desired configuration.

16. The breathing conduit of claim 15, wherein at least said section is at least partially longitudinally collapsible and at least partially longitudinally expandable.

17. The breathing conduit of claim 16, wherein at least said section comprises corrugated tubing.

18. The breathing conduit of claim 15, wherein at least said section carries at least one elongate compliant member that is configured to be bent to shape at least said section into said desired configuration and maintain said desired configuration.

19. A method for obtaining a resting metabolic rate of an individual, comprising:
placing the individual in a resting position;
coupling a breathing apparatus and conduit in communication therewith in flow communication between an airway of the individual and an apparatus for monitoring the individual's respiration; and
manipulating at least a portion said conduit into a desired configuration, said conduit being configured so as to substantially maintain said desired configuration.

20. The method of claim 19, wherein said coupling comprises:
coupling said breathing apparatus in substantially fluid-tight connection to at least a mouth of the individual; and
coupling said conduit in substantially fluid tight communication to said apparatus.

21. The method of claim 19, wherein said manipulating comprises at least one of at least partially longitudinally collapsing locations of at least said portion and at least partially longitudinally expanding locations of at least said portion.

22. The method of claim 19, wherein said manipulating comprises bending at least one elongate compliant member carried upon a wall of at least said portion.

23. The method of claim 19, wherein, upon said manipulating, said conduit at least partially supports said breathing apparatus.

24. The method of claim 19, further comprising substantially restricting respiration through a nose of the individual.
